SEQUENCE LISTING (26 sequences)

SEQ ID NO: 1 - DNA, 1329 bp, Listeria monocytogenes
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgag

SEQ ID NO: 2 - protein, 442 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDL

SEQ ID NO: 3 - protein, 14 aa, Listeria monocytogenes
KTEEQPSEVNTGPR

SEQ ID NO: 4 - protein, 28 aa, Listeria monocytogenes
KASVTDTSEGDLDSSMQSADESTPQPLK

SEQ ID NO: 5 - protein, 20 aa, Listeria monocytogenes
KNEEVNASDFPPPPTDEELR

SEQ ID NO: 6 - protein, 33 aa, Listeria monocytogenes
RGGIPTSEEFSSLNSGDFTDDENSETTEEEIDR

SEQ ID NO: 7 - protein, 17 aa, Streptococcus pyogenes
KQNTASTETTTTNEQPK

SEQ ID NO: 8 - protein, 17 aa, Streptococcus equisimilis
KQNTANTETTTTNEQPK

SEQ ID NO: 9 - DNA, 28 bp, Artificial Sequence
  Lm-LLO-ISG15 forward primer
taatctcgagatggcctgggacctaaag

SEQ ID NO: 10 - DNA, 28 bp, Artificial Sequence
  Lm-LLO-ISG15 reverse primer
attaactagtttaggcacactggtcccc

SEQ ID NO: 11 - DNA, 18 bp, Artificial Sequence
  ISG15 forward primer
atggcctgggacctaaag

SEQ ID NO: 12 - DNA, 18 bp, Artificial Sequence
  ISG15 reverse primer
ttaggcacactggtcccc

SEQ ID NO: 13 - DNA, 20 bp, Artificial Sequence
  18SRNA forward primer
cggctaccacatccaaggaa

SEQ ID NO: 14 - DNA, 18 bp, Artificial Sequence
  18SRNA reverse primer
gctggaattaccgcggct

SEQ ID NO: 15 - DNA, 21 bp, Artificial Sequence
  ACTIN forward primer
gtgggccgctctaggcaccaa

SEQ ID NO: 16 - DNA, 24 bp, Artificial Sequence
  Actin reverse primer
ctctttgatgtcacgcacgatttc

SEQ ID NO: 17 - protein, 9 aa, Artificial Sequence
  HIV-gag H-2Kd CTL epitope
AMQMLKETI

SEQ ID NO: 18 - protein, 9 aa, Artificial Sequence
  ISG15 d1 primer
RGHSNIYEV

SEQ ID NO: 19 - protein, 9 aa, Artificial Sequence
  ISG15 d2 primer
LGPSSTVML

SEQ ID NO: 20 - protein, 9 aa, Artificial Sequence
  HIV-gag H-2Kd CTL epitope peptide
AMQMLKETI

SEQ ID NO: 21 - protein, 9 aa, Artificial Sequence
  ISG15-d1 primer
RGHSNIYEV

SEQ ID NO: 22 - protein, 9 aa, Artificial Sequence
  ISG15-d2 primer
LGPSSTVML

SEQ ID NO: 23 - protein, 9 aa, Artificial Sequence
  HIV-gag H-2Kd CTL epitope peptide
AMQMLKETI

SEQ ID NO: 24 - protein, 9 aa, Artificial Sequence
  Her2/neu-specific H-2Kd epitope peptides Her2-EC1
PYNYLSTEV

SEQ ID NO: 25 - protein, 10 aa, Artificial Sequence
  Her2-EC2
LFRNPHQALL

SEQ ID NO: 26 - protein, 9 aa, Artificial Sequence
  Her2-IC1
PYVSRLLGI

(19) United States
(12) Patent Application Publication
(10) Pub. No.: US 2011/0305724 A1
(43) Pub. Date: Dec. 15, 2011

(54) IMMUNOTHERAPEUTIC, ANTI-TUMORIGENIC COMPOSITIONS AND METHODS OF USE THEREOF

(76) Inventors: Yvonne Paterson, Philadelphia, PA (US); Laurence Wood, Philadelphia, PA (US)

(21) Appl. No.: 13/090,159

(22) Filed: Apr. 19, 2011

Related U.S. Application Data
(60) Provisional application No. 61/325,473, filed on Apr. 19, 2010.

(51) Int. Cl. A61K 39/02; C07K 2/00; A61P 37/04; A61P 35/00; A61P 35/04; C12N 15/62; A61K 31/711

(52) U.S. Cl. 424/200.1; 536/23.4; 530/350; 514/44 R

(57) ABSTRACT

This invention relates to immunotherapeutic vaccine composition comprising an ISG15 tumor antigen and to methods of preventing and treating a tumor growth by using said immunotherapeutic composition.

### CROSS-REFERENCE TO RELATED APPLICATIONS

This application claims priority of U.S. Provisional Application Ser. No. 61/325,473, filed 10 Apr. 2010. This application is hereby incorporated in its entirety by reference herein.

### GOVERNMENT INTEREST

This invention was supported, in part, by Grant Number CA101968-05 and CA109253-03 from the NIH. The United States government may have certain rights in the invention.

### FIELD OF INVENTION

This invention is directed to immunotherapeutic vaccine composition comprising an ISG15 tumor antigen and to methods of preventing and treating a tumor growth by using said immunotherapeutic composition.

### BACKGROUND OF THE INVENTION

In recent years, immunotherapy has proven to be a viable option in the treatment of breast cancer. While only passive immunotherapy is widely used in the clinic at the moment, active therapeutic vaccination is achieving increased effectiveness in the clinic and in preclinical models of cancer. ISG15, a ubiquitin-like protein, is over-expressed in human breast cancer cell lines and breast cancer tissue. Lm vaccines induce a strong CTL response against a target antigen when fused to non-hemolytic Listeriolysin O (LLO) and secreted by recombinant Lm vaccines.

There exists an ongoing need for vaccines that can stimulate a general immune response against cancer cells, such as breast cancer cells, to prevent and treat tumor growth and metastases. The present invention fullfills this need by providing an immunotherapeutic vaccine composition encoding a ISG15 tumor antigen.

In one embodiment, the invention relates to a recombinant Listeria vaccine vector comprising a recombinant nucleic acid encoding a recombinant polypeptide, wherein the recombinant polypeptide comprises a non-hemolytic N-terminal Listeriolysin (LLO) fused to a tumor antigen, and wherein the tumor antigen is ISG15 or an immunogenic fragment thereof.

In another embodiment, the invention relates to a method of diagnosing a tumor growth in a subject, the method comprising the step of obtaining a biological sample from the subject and measuring the expression level of an ISG15 antigen in the biological sample, wherein when the ISG15 expression level is elevated in the subject over ISG15 levels observed obtained from a pool of normal subjects, the subject is effectively diagnosed as having a tumor growth.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1. Elevated expression of ISG15 in mouse mammary tumors. (A) mRNA was extracted from autochthonous mouse mammary tumors (n=9) from FVB/N HER2/neu transgenic mice and normal mammary tissues (n=4) from FVB/N mice. After cDNA conversion, qPCR analysis was performed to determine relative ISG15 mRNA expression. (B) Western blot analysis of tissue lysates from normal mammary tissue and HER2/neu mammary tumor tissues with anti-ISG15 antibody, top panel, and anti-GAPDH antibody to demonstrate equivalent protein loading, bottom panel. (C) qPCR of cDNA from mammary tumor cell lines NT2 and 4T1-Luc were compared against normal mammary tissue and non-transformed cell line NIH-3T3 for expression of ISG15 mRNA (n=3). (D) qPCR analysis of ISG15 expression in a panel of normal tissues (n=3) compared to autochthonous mammary tumors from HER2/neu transgenic mice (n=7).

FIG. 2. Construction of a Listeria-based CTL vaccine against ISG15. (A) Illustration depicting the Listeria expression vector, pGG34-LLO-ISG15, that was electroporated into the prfA⁻ XFL7 Listeria strain to construct the attenuated Listeria vaccine, Lm-LLO-ISG15. (B) Western blot analysis of TCA-precipitated proteins from the media of Lm-LLO-ISG15 and control Lm vaccine, Lm-LLO-OVA, cultures. Precipitated proteins were subjected to SDS-PAGE and western blot analysis with antibodies against mouse ISG15 (top panel), chicken ovalbumin (middle panel), and Listeriolysin O (bottom panel). (C) ELISpot analysis of ISG15-specific IFNγ responses from splenocytes of 8-week old Balb/c mice that were vaccinated i.p. twice with either Lm-LLO-ISG15 or control Lm. Results are depicted as IFNγ-secreting SFCs per 2×10⁶ splenocytes. (D) Number of pups per litter for female mice vaccinated with either a control Lm vaccine (2×10⁸ CFU) or Lm-LLO-ISG15 (2×10⁸ CFU). (E) Mean pup weight of littermates from each vaccinated group of females on day one post-birth depicted in grams.

FIG. 3. Therapeutic impact on mouse mammary tumors after Lm-LLO-ISG15 vaccination. (A) Tumor load study to determine the effectiveness of Lm-LLO-ISG15 against implanted NT2 mammary tumors. NT2 tumor cells were implanted s.c. in the hind flank of FVB/N mice and subsequently vaccinated with Lm-LLO-ISG15 or control Lm. Tumor size was monitored with calipers until experiment end and tumor volume calculated. (B) Tumor load study to determine the ability of Lm-LLO-ISG15 vaccination to control the growth of implanted primary 4T1-Luc mammary tumors. 4T1-Luc tumor cells were implanted in the mammary tissue of Balb/c mice and mice were subsequently vaccinated with Lm-LLO-ISG15 or control Lm. (C) Metastatic tumor study to determine the ability of Lm-LLO-ISG15 vaccination to control metastatic spread of 4T1-Luc after implantation in the mammary gland. Briefly, 4T1-Luc cells are implanted into the mammary tissue of Balb/c mice and mice are subsequently vaccinated with Lm-LLO-ISG15 or control Lm. After 32 days post implantation, lungs from vaccinated tumor-bearing mice are removed and perfused with PBS. Lung surface metastatic nodules were then counted with a light microscope.

FIG. 4. Delayed progression of HER2/neu+autochthonous mammary tumors and epitope spreading by Lm-LLO-ISG15. (A) The FVB/N Her2/neu transgenic mouse model was used to determine if Lm-LLO-ISG15 vaccination can delay autochthonous mammary tumor progression in comparison to control Lm vaccination. FVB/N HER2/neu transgenic mice were injected six times with either Lm-LLO-ISG15 (2×10⁸ CFU) or the control Lm vaccine, Lm-LLO-OVA (2×10⁸ CFU), starting at 6 wk of age and continued every 3 weeks until week 21. Tumor incidence was monitored on a weekly basis. (B) ELISpot analysis of ISG15-specific IFN-γ responses in the spontaneous breast tumors from naive mice. After allowing for tumor formation, tumor-bearing mice were vaccinated twice (day 0 and 7) with Control Lm and Lm-LLO-ISG15 followed by removal of tumors and ELISpot analysis on day 14. (C) ELISpot analysis demonstrating epitope spreading to HER2/neu in splenocytes of Lm-LLO-ISG15 vaccinated NT2 tumor-bearing FVB/N HER2/neu transgenic mice at the completion of the experiment. (D) TIL tetramer analysis demonstrating an increased percentage of HER2/neu-specific CD8+62L- in the tumors of Lm-LLO-ISG15 vaccinated 4T1-Luc tumor bearing mice in comparison to control Lm vaccinated mice.

FIG. 5. Therapeutic impact of ISG15 vaccination is CD8-dependent. (A) CD8 depletion experiment of 4T1-Luc tumor-bearing mice. Briefly, Balb/c mice were implanted with 4T1-Luc tumor cells and depleted of CD8⁺ cells or mock depleted in addition to vaccination with Lm-LLO-ISG15 or control Lm. (B) Winn assay performed to measure direct cytolytic activity of Lm-LLO-ISG15 CD8-enriched splenocytes. CD4-depleted splenocytes from Lm-LLO-ISG15 or control Lm vaccinated mice were mixed with 4T1-Luc cells and implanted in naïve Balb/c mice. (C) Graph depicting percent tumor-free survival of Balb/c mice from the experiment depicted in FIG. 5B.

FIG. 6. Expansion of ISG15-specific CTL clones in vivo results in anti-tumor responses. After implantation of 4T1-Luc tumor cells in the mammary tissue of female Balb/c mice, mice were subsequently vaccinated with PBS or CpG along with either a control or an ISG15 epitope peptide. (A) Tumor volume for each group was measured throughout the course of the experiment. (B) At the conclusion of the experiment, primary tumors were removed and mean tumor mass for each vaccinated group was calculated. (C) Additionally, lungs from mice of each vaccinated group were also removed at the conclusion of the experiment for inspection of surface metastases. Mean number of lung surface metastases was calculated for vaccinated group. (D) ELISpot analysis of ISG15 d1-specific IFN-γ responses by tumor-infiltrating lymphocytes (TILs) from PBS and pISG15 d1/CPG vaccinated mice. (E) ELISpot analysis of ISG15 d2-specific IFN-γ responses by TILs from PBS and pISG15 d2/CPG vaccinated mice.

### DETAILED DESCRIPTION OF THE INVENTION

This invention provides in one embodiment a recombinant Listeria vaccine vector comprising a recombinant nucleic acid encoding a recombinant polypeptide, wherein the recombinant polypeptide comprises a non-hemolytic N-terminal Listeriolysin (LLO) fused to a tumor antigen, and wherein the tumor antigen is ISG15 or a functional fragment thereof. In another embodiment, the tumor antigen is ISG15 or an immunogenic fragment thereof.

In another embodiment, the present invention a recombinant nucleic acid molecule encoding the fusion polypeptide provided herein

In another embodiment, the present invention provides a recombinant polypeptide encoded by the recombinant nucleic acid molecule provided herein.

In another embodiment, provided herein is a method of diagnosing a tumor growth in a subject, the method comprising the step of obtaining a biological sample from the subject and measuring the expression profile of an ISG15 antigen in the biological sample, wherein when the ISG15 expression level is observed to be elevated in the subject over the levels observed in that of a control sample the subject is effectively diagnosed as having a ISG15-expressing tumor growth.

In another embodiment, provided herein is a method of monitoring a tumor growth in a subject, the method comprising the step of obtaining a biological sample from the subject and measuring the expression profile of an ISG15 antigen in the biological sample, wherein measuring the ISG15 expression level in the subject over the levels observed in that of a control sample enables the monitoring of a tumor growth in the subject. In another embodiment, the biological sample is tissue, blood, urine, semen, sputa, spinal chord fluid.

The measurement of the ISG15 expression profile can be carried out by any assay used for measuring expression levels of a marker which includes but is not limited to immunoassays (such as various ELISAs), immunoblots, immunohistochemical assays, flourescence-based assays, quantitative HPLC alone or in combination with mass spectrometry, or any other assay known in the art, as will be understood by a skilled artisan. The measured expression profile can then be compared with a control profile to effectively diagnose a tumor growth in a subject.

The method of diagnosing an ISG15 tumor growth can be further validated by diagnosing the tumor growth through other means known in the art, which include but is not limited to identification of a different tumor antigen or through more routine methods involving scanning procedures such as MRIs, PET-Scans, mammographies, and the like.

The ISG15 provided herein can be any ISG15 available in the art, including, but not limited to the following provided by accession numbers AAH09507.1, CAI15574.1, EDL15082.1, AAH31424.1, AAH83156.1, AAI09347.1.

In another embodiment, a ISG15 protein or antigen is also referred to as “ISG15 ubiquitin-like modifier”, “UCRP”, “IFI15”, “G1P2”.

In another embodiment, provided here is a recombinant nucleic acid molecule encoding the tumor antigen provided herein. In another embodiment, the recombinant nucleic acid molecule further encodes a fusion protein comprising a non-hemolytic Listeriolysin O (LLO) protein genetically fused to the antigen.

The term “Nucleic acid molecule” refers, in one embodiment, to a plasmid. In another embodiment, the term refers to an integration vector. In another embodiment, the term refers to a plasmid comprising an integration vector. In another embodiment, the integration vector is a site-specific integration vector. In another embodiment, a nucleic acid molecule of methods and compositions of the present invention is composed of any type of nucleotide known in the art. Each possibility represents a separate embodiment of the present invention.

In one embodiment, the nucleic acid molecule provided herein is used to transform the Listeria in order to arrive at a recombinant Listeria. In another embodiment, provided herein is a recombinant Listeria vaccine strain comprising a nucleic acid molecule, wherein the nucleic acid molecule comprises a first open reading frame encoding a polypeptide, wherein the polypeptide comprises a ISG15 antigen, wherein the nucleic acid molecule further comprises a second and/or a third open reading frame each encoding a metabolic enzyme, and wherein the metabolic enzyme complements an endogenous gene that is lacking in the chromosome of said recombinant Listeria strain. In one embodiment, the nucleic acid molecule is integrated into the Listeria genome. In another embodiment, the nucleic acid molecule is in a plasmid in the recombinant Listeria vaccine strain. In yet another embodiment, the plasmid is stably maintained in the recombinant Listeria vaccine strain in the absence of antibiotic selection. In another embodiment, the plasmid does not confer antibiotic resistance upon the recombinant Listeria. In another embodiment, the recombinant Listeria strain is attenuated. In another embodiment, the recombinant Listeria is an attenuated auxotrophic strain. In another embodiment, the high metabolic burden that the expression of a foreign antigen exerts on a bacterium such as one of the present invention is also an important mechanism of attenuation. In one embodiment the attenuated strain is Lmdda. In another embodiment, the recombinant Listeria provided herein lacks an actA gene. In another embodiment, it lacks an Internalin C gene. In another embodiment the recombinant Listeria lacks both an internalin C and an actA gene.

In another embodiment, the nucleic acid provided herein used to transform Listeria lacks a virulence gene. In another embodiment, the nucleic acid molecule integrated into the Listeria genome carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is a PrfA gene. As will be understood by a skilled artisan, the virulence gene can be any gene known in the art to be associated with virulence in the recombinant Listeria.

The term “Stably maintained” refers, in another embodiment, to maintenance of a nucleic acid molecule or plasmid in the absence of selection (e.g. antibiotic selection) for 10 generations, without detectable loss. In another embodiment, the period is 15 generations to 20 generations. In another embodiment, the period is 21 to 30 generations. In another embodiment, the period is 31 to 40 generations. In another embodiment, the period is 41 to 60 generations. In another embodiment, the period is 61 to 80 generations. In another embodiment, the period is 81 to 100 generations. In another embodiment, the period is 101 to 150 generations. In another embodiment, the period is 151 to 200 generations. In another embodiment, the period is 201 to 250 generations. In another embodiment, the period is 251 to 300 generations. In another embodiment, the period is 15 generations. In another embodiment, the period is 301 to 400 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro. Each possibility represents a separate embodiment of the present invention.

In another embodiment, the plasmid provided herein is stably maintained in a host cell.

In one embodiment, provided herein is a recombinant polypeptide encoded by the recombinant nucleic acid molecule. In another embodiment, the recombinant polypeptide comprises a recombinant non-hemolytic LLO fused to the tumor antigen provided herein.

In one embodiment, the fusion protein of the methods and compositions of the present invention comprises an LLO signal sequence from LLO. In another embodiment, the two molecules of the protein (the LLO fragment and the antigen) are joined directly. In another embodiment, the two molecules are joined by a short spacer peptide, consisting of one or more amino acids. In one embodiment, the spacer has no specific biological activity other than to join the proteins or to preserve some minimum distance or other spatial relationship between them. In another embodiment, the constituent amino acids of the spacer are selected to influence some property of the molecule such as the folding, net charge, or hydrophobicity. In another embodiment, the two molecules of the protein (the LLO fragment and the antigen) are synthesized separately or unfused. In another embodiment, the two molecules of the protein are synthesized separately from the same nucleic acid. In yet another embodiment, the two molecules are individually synthesized from separate nucleic acids. Each possibility represents a separate embodiment of the present invention.

In one embodiment, the immunotherapeutic vaccine composition provided herein comprises a delivery vector. It is to be understood that the delivery vector can be any such vector known in the art, including, but not limited to, a plasmid, a live bacteria, or a combination thereof. In a preferred embodiment, the delivery vector is a live bacteria and in another embodiment, it is a live Listeria strain. In another embodiment, the Listeria strain is Listeria monocytogenes (LM). In another embodiment, the Listeria is Listeria ivanovii. In another embodiment, the Listeria is Listeria welshimeri. In another embodiment, the Listeria is Listeria seeligeri. Each type of Listeria represents a separate embodiment of the present invention.

In one embodiment, provided herein are compositions and methods for preventing or treating a tumor growth. In another embodiment, the “tumor growth” is a benign tumor growth, a malignant tumor growth or a cancer. In one embodiment, the cancer treated by a method of the present invention includes, but is not limited to, cervical cancer, a breast cancer. In a ISG15 containing cancer, an Her2 containing cancer, a melanoma, a pancreatic cancer, an ovarian cancer, a gastric cancer, a carcinomatous lesion of the pancreas, a pulmonary adenocarcinoma, a colorectal adenocarcinoma, a pulmonary squamous adenocarcinoma, a gastric adenocarcinoma, an ovarian surface epithelial neoplasm (e.g. a benign, proliferative or malignant variety thereof), an oral squamous cell carcinoma, a non small-cell lung carcinoma, an endometrial carcinoma, a bladder cancer, a head and neck cancer, a prostate carcinoma, or a combination thereof. Each possibility represents a separate embodiment of the present invention.

In one embodiment, the present invention provides a recombinant Listeria strain expressing the antigen. The present invention also provides recombinant peptides comprising a listeriolysin O (LLO) protein fragment fused to a ISG15 protein or fragment thereof, vaccines and immunogenic compositions comprising same, and methods of inducing an anti-ISG15 immune response and treating and vaccinating against a ISG15-expressing tumor, comprising the same.

In another embodiment, the present invention provides a recombinant Listeria strain expressing the antigen. In a related aspect the invention also provides recombinant peptides comprising a listeriolysin (LLO) protein fragment fused to a ISG15 protein or fragment thereof, vaccines and immunogenic compositions comprising same, and methods of inducing an immune response against an antigen that is not ISG15. In another embodiment, the invention provides methods of inducing an anti-Her2/neu immune response and treating and vaccinating against a Her2/neu-expressing tumor, comprising the same. This is accomplished via the phenomenom of epitope spreading.

In one embodiment, the present invention provides a method for “epitope spreading” of a tumor. In another embodiment, the immunization using the compositions and methods provided herein induce epitope spreading onto tumor antigens other than the antigen carried in the vaccine of the present invention.

In one embodiment, vaccination with recombinant antigen-expressing LM induces epitope spreading. In another embodiment, vaccination with LLO-antigen fusions, even outside the context of ISG15, induces epitope spreading as well. Each possibility represents a separate embodiment of the present invention.

In one embodiment, the polypeptide provided herein is a fusion protein comprising an additional polypeptide selected from the group consisting of: a) non-hemolytic LLO protein or N-terminal fragment, b) a PEST sequence, or c) an ActA fragment, and further wherein said additional polypeptide is fused to said ISG15 antigen. In another embodiment, the additional polypeptide is functional. In another embodiment, a fragment of the additional polypeptide is immunogenic. In another embodiment, the additional polypeptide is immunogenic.

The LLO utilized in the methods and compositions provided herein is, in one embodiment, a Listeria LLO. In one embodiment, the Listeria from which the LLO is derived is Listeria monocytogenes (LM). In another embodiment, the Listeria is Listeria ivanovii. In another embodiment, the Listeria is Listeria welshimeri. In another embodiment, the Listeria is Listeria seeligeri. In another embodiment, the LLO protein is a non-Listeria1 LLO protein.

In one embodiment, the LLO protein is encoded by the following nucleic acid sequence set forth in (SEQ ID NO:1)

(SEQ ID NO: 1)
atgaaaaaaataatgctagtttttattacacttatattagttagtctaccaattgcgcaacaaactgaagcaaaggatgcatctgcattcaa
taaagaaaattcaatttcatccatggcaccaccagcatctccgcctgcaagtcctaagacgccaatcgaaaagaaacacgcggatgaaatc
gataagtatatacaaggattggattacaataaaaacaatgtattagtataccacggagatgcagtgacaaatgtgccgccaagaaaaggt
tacaaagatggaaatgaatatattgagtggagaaaaagaagaaatccatcaatcaaaataatgcagacattcaagagtgaatgcaatttc
gagcctaacctatccaggtgctctcgtaaaagcgaattcggaattagtagaaaatcaaccagatgttctccctgtaaaacgtgattcattaa
cactcagcattgatttgccaggtatgactaatcaagacaataaaatagttgtaaaaaatgccactaaatcaaacgttaacaacgcagtaaat
acattagtggaaagatggaatgaaaaatatgctcaagcttatccaaatgtaagtgcaaaaattgattatgatgacgaaatggcttacagtg
aatcacaattaattgcgaaatttggtacagcatttaaagctgtaaataatagcttgaatgtaaacttcggcgcaatcagtgaagggaaaatg
caagaagaagtcattagttttaaacaaatttactataacgtgaatgttaatgaacctacaagaccttccagattttttcggcaaagctgttac
taaagagcagttgcaagcgcttggagtgaatgcagaaaatcctcctgcatatatctcaagtgtggcgtatggccgtcaagtttatttgaaatta
tcaactaattcccatagtactaaagtaaaagctgcttttgatgctgccgtaagcggaaaatctgtctcaggtgatgtagaactaacaaatatc
atcaaaaattcttccttcaaagccgtaatttacggaggttccgcaaaagatgaagttcaaatcatcgacggcaacctcggagacttacgcg
atattttgaaaaaaggcgctacttttaatcgagaaacaccaggagttcccattgcttatacaacaaacttcctaaaagacaatgaattagctg
ttattaaaaacaactcagaatatattgaaacaacttcaaaagcttatacagatggaaaaattaacatcgatcactctggaggatacgttgctc
aattcaacatttcttgggatgaagtaaattatgatctcgag.

In another embodiment, the LLO protein has the sequence SEQ ID NO:2

(SEQ ID NO: 2)
M K K I M L V F I T L I L V S L P I A Q Q T E A K D A S A F N K E
N S I S S M A P P A S P P A S P K T P I E K K H A D E I D K Y I Q
G L D Y N K N N V L V Y H G D A V T N V P P R K G Y K D G N E
Y I V V E K K K K S I N Q N N A D I Q V V N A I S S L T Y P G A L
V K A N S E L V E N Q P D V L P V K R D S L T L S I D L P G M T
N Q D N K I V V K N A T K S N V N N A V N T L V E R W N E K Y
A Q A Y P N V S A K I D Y D D E M A Y S E S Q L I A K F G T A F
K A V N N S L N V N F G A I S E G K M Q E E V I S F K Q I Y Y N
V N V N E P T R P S R F F G K A V T K E Q L Q A L G V N A E N P
P A Y I S S V A Y G R Q V Y L K L S T N S H S T K V K A A F D A
A V S G K S V S G D V E L T N I I K N S S F K A V I Y G G S A K D
E V Q I I D G N L G D L R D I L K K G A T F N R E T P G V P I A Y
T T N F L K D N E L A V I K N N S E Y I E T T S K A Y T D G K I N
I D H S G G Y V A Q F N I S W D E V N Y D L

The first 25 amino acids of the proprotein corresponding to this sequence are the signal sequence and are cleaved from LLO when it is secreted by the bacterium. Thus, in this embodiment, the full length active LLO protein is 504 residues long. In another embodiment, the LLO protein has a sequence set forth in GenBank Accession No. DQ054588, DQ054589, AY878649, U25452, or U25452. In another embodiment, the LLO protein is a variant of an LLO protein. In another embodiment, the LLO protein is a homologue of an LLO protein. Each possibility represents a separate embodiment of the present invention.

In another embodiment, “truncated LLO” or “tLLO” refers to a fragment of LLO that comprises the PEST-like domain. In another embodiment, the terms refer to an LLO fragment that does not contain the activation domain at the amino terminus and does not include cystine 484. In another embodiment, the LLO fragment consists of a PEST sequence. In another embodiment, the LLO fragment comprises a PEST sequence. In another embodiment, the LLO fragment consists of about the first 400 to 441 amino acids of the 529 amino acid full-length LLO protein. In another embodiment, the LLO fragment is a non-hemolytic form of the LLO protein.

In another embodiment of methods and compositions of the present invention, the fusion protein comprises the ISG15 antigen or a functional fragment thereof and an additional polypeptide. In one embodiment, the additional polypeptide is a non-hemolytic LLO protein or fragment thereof (Examples herein). In another embodiment, the additional polypeptide is a PEST sequence. In another embodiment, the additional polypeptide is an ActA protein or a fragment thereof. ActA proteins and fragments thereof augment antigen presentation and immunity in a similar fashion to LLO.

In one embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425. Each possibility represents a separate embodiment of the present invention.

In another embodiment, a fusion protein of methods and compositions of the present invention comprises a PEST sequence, either from an LLO protein or from another organism, e.g. a prokaryotic organism.

The PEST-like AA sequence has, in another embodiment, a sequence selected from SEQ ID NO: 3-7. In another embodiment, the PEST-like sequence is a PEST-like sequence from the LM ActA protein. In another embodiment, the PEST-like sequence is KTEEQPSEVNTGPR (SEQ ID NO: 3), KASVTDTSEGDLDSSMQSADESTPQPLK (SEQ ID NO: 4), KNEEVNASDFPPPPTDEELR (SEQ ID NO: 5), or RGGIPTSEEFSSLNSGDFTDDENSETTEEEIDR (SEQ ID NO: 6). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 7) at AA 35-51. In another embodiment, the PEST-like sequence is from Streptococcus equisimilis Streptolysin O, e.g. KQNTANTETTTTNEQPK (SEQ ID NO: 8) at AA 38-54. In another embodiment, the PEST-like sequence is another PEST-like AA sequence derived from a prokaryotic organism. In another embodiment, the PEST-like sequence is any other PEST-like sequence known in the art. Each possibility represents a separate embodiment of the present invention.

In one embodiment, fusion of an antigen to the PEST-like sequence of LM enhances cell mediated and anti-tumor immunity of the antigen. Thus, fusion of an antigen to other PEST-like sequences derived from other prokaryotic organisms will also enhance immunogenicity of the antigen. PEST-like sequence of other prokaryotic organism can be identified in accordance with methods such as described by, for example Rechsteiner and Rogers (1996, Trends Biochem. Sci. 21:267-271) for LM. Alternatively, PEST-like AA sequences from other prokaryotic organisms can also be identified based by this method. Other prokaryotic organisms wherein PEST-like AA sequences would be expected to include, but are not limited to, other Listeria species. In another embodiment, the PEST-like sequence is embedded within the antigenic protein. Thus, in another embodiment, “fusion” refers to an antigenic protein comprising both the antigen and the PEST-like amino acid sequence either linked at one end of the antigen or embedded within the antigen.

In another embodiment, provided herein is a vaccine comprising a recombinant polypeptide of the present invention.

In another embodiment, provided herein is a nucleotide molecule encoding a recombinant polypeptide of the present invention. In another embodiment, provided herein is a vaccine comprising the nucleotide molecule.

In another embodiment, provided herein is a nucleotide molecule encoding a recombinant polypeptide of the present invention.

In another embodiment, provided herein is a recombinant polypeptide encoded by the nucleotide molecule of the present invention.

In another embodiment, provided herein is a vaccine comprising a nucleotide molecule or recombinant polypeptide of the present invention.

In another embodiment, provided herein is an immunogenic composition comprising a nucleotide molecule or recombinant polypeptide of the present invention.

In another embodiment, provided herein is a vector comprising a nucleotide molecule or recombinant polypeptide of the present invention.

Fusion proteins comprising the ISG15 antigen may be prepared by any suitable method, including, for example, cloning and restriction of appropriate sequences or direct chemical synthesis by methods discussed below. Alternatively, subsequences may be cloned and the appropriate subsequences cleaved using appropriate restriction enzymes. The fragments may then be ligated to produce the desired DNA sequence. In one embodiment, DNA encoding the antigen can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Each method represents a separate embodiment of the present invention.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed by any other method known in the art.

In one embodiment, provided herein is a method of treating, suppressing, or inhibiting a cancer or a tumor growth in a subject, whereby and in another embodiment, said cancer is associated with expression of an antigen or fragment thereof comprised in the composition of the present invention. In another embodiment, the method comprises administering to said subject a composition comprising the recombinant polypeptide, recombinant Listeria, or recombinant vector of the present invention. In yet another embodiment, the subject mounts an immune response against the antigen-expressing cancer or the antigen-expressing tumor, thereby treating, suppressing, or inhibiting a cancer or a tumor growth in a subject.

In another embodiment, provided herein is a method of treating, suppressing, or inhibiting a cancer or a tumor growth in a subject by epitope spreading whereby and in another embodiment, said cancer is associated with expression of an antigen or fragment thereof comprised in the composition of the present invention. In another embodiment, the method comprises administering to said subject a composition comprising the recombinant polypeptide, recombinant Listeria, or recombinant vector of the present invention. In yet another embodiment, the subject mounts an immune response against the antigen-expressing cancer or the antigen-expressing tumor, thereby treating, suppressing, or inhibiting a cancer or a tumor growth in a subject.

In one embodiment, provided herein is a method of enhancing an anti-ISG15 immune response in a subject, the method comprising the step of administering to the subject a therapeutically effective dose of the an immunotherapeutic vaccine composition provided herein.

Methods of measuring immune responses are well known in the art, and include, e.g. measuring suppression of tumor growth, flow cytometry, target cell lysis assays (e.g. chromium release assay), the use of tetramers, and others. Each method represents a separate embodiment of the present invention.

The antigen in methods and compositions of the present invention is, in one embodiment, expressed at a detectable level on a non-tumor cell of the subject. In another embodiment, the antigen is expressed at a detectable level on at least a certain percentage (e.g. 0.01%, 0.03%, 0.1%, 0.3%, 1%, 2%, 3%, or 5%) of non-tumor cells of the subject. In one embodiment, “non-tumor cell” refers to a cell outside the body of the tumor. In another embodiment, “non-tumor cell” refers to a non-malignant cell. In another embodiment, “non-tumor cell” refers to a non-transformed cell. In another embodiment, the non-tumor cell is a somatic cell. In another embodiment, the non-tumor cell is a germ cell. Each possibility represents a separate embodiment of the present invention.

“Detectable level” refers, in one embodiment, to a level detectable by a standard assay. In one embodiment, the assay is an immunological assay. In one embodiment, the assay is enzyme-linked immunoassay (ELISA). In another embodiment, the assay is Western blot. In another embodiment, the assay is FACS. It is to be understood by a skilled artisan that any other assay available in the art can be used in the methods provided herein. In another embodiment, a detectable level is determined relative to the background level of a particular assay. Methods for performing each of these techniques are well known to those skilled in the art, and each technique represents a separate embodiment of the present invention.

In another embodiment of the methods of the present invention, the subject mounts an immune response against the antigen-expressing tumor or target antigen, thereby mediating the anti-tumor effects.

In another embodiment, provided herein is a method of eliciting an anti-ISG15 adaptive immune response in a subject, the method comprising the step of administering to the subject a therapeutically effective dose of the recombinant Listeria vaccine vector provided herein.

In another embodiment, provided herein is a method of treating a tumor growth in a subject, the method comprising the step of administering to the subject a therapeutically effective dose of recombinant Listeria vaccine vector provided herein.

In another embodiment, provided herein is a method of treating a ISG15 antigen-expressing tumor growth in a subject, the method comprising the step of administering to the subject a therapeutically effective dose of the recombinant Listeria vaccine vector provided herein.

In another embodiment, provided herein is a method of treating an Her-2/neu expressing tumor growth in a subject, the method comprising the step of administering to the subject a therapeutically effective dose of the recombinant Listeria vaccine vector provided herein.

In another embodiment, provided herein is a method of treating a metastases in a subject, the method comprising the step of administering to said subject a therapeutically effective dose of an the recombinant Listeria vaccine vector provided herein.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to inducing remission. In another embodiment, “treating” refers to slowing the progression of a disease. The terms “reducing”, “suppressing” and “inhibiting” refer in another embodiment to lessening or decreasing. Each possibility represents a separate embodiment of the present invention.

In another embodiment, provided herein is a method of preventing the onset of a tumor in a subject, the method comprising the step of administering to the subject a therapeutically effective dose of the recombinant Listeria vaccine vector provided herein.

In another embodiment, provided herein is a method of preventing the onset of a ISG15 antigen-expressing tumor in a subject, the method comprising the step of administering to the subject a therapeutically effective dose of the recombinant Listeria vaccine vector provided herein.

In another embodiment, provided herein is a method of preventing the onset of a Her2/neu antigen-expressing tumor in a subject, the method comprising the step of administering to the subject a therapeutically effective dose of the recombinant Listeria vaccine vector provided herein.

In another embodiment, provided herein is a method of preventing metastatic tumor growth in a subject, the method comprising the step of administering to the subject a therapeutically effective dose of the recombinant Listeria vaccine vector provided herein.

In another embodiment, provided herein is a method of delaying progression of spontaneous breast tumors in a subject, the method comprising the step of administering to the subject a therapeutically effective dose of the recombinant Listeria vaccine vector provided herein.

In another embodiment, the immune response elicited by methods and compositions of the present invention comprises a CD8⁺ T cell-mediated response. In another embodiment, the immune response consists primarily of a CD8⁺ T cell-mediated response. In another embodiment, the only detectable component of the immune response is a CD8⁺ T cell-mediated response.

In another embodiment, the immune response elicited by methods and compositions provided herein comprises a CD4⁺ T cell-mediated response. In another embodiment, the immune response consists primarily of a CD4⁺ T cell-mediated response. In another embodiment, the only detectable component of the immune response is a CD4⁺ T cell-mediated response. In another embodiment, the CD4⁺ T cell-mediated response is accompanied by a measurable antibody response against the antigen. In another embodiment, the CD4⁺ T cell-mediated response is not accompanied by a measurable antibody response against the antigen.

In another embodiment, the present invention provides a method of inducing a CD8³⁰ T cell-mediated immune response in a subject against a subdominant CD8⁺ T cell epitope of an antigen, comprising the steps of (a) fusing a nucleotide molecule encoding the Her2-neu chimeric antigen or a fragment thereof to a nucleotide molecule encoding an N-terminal fragment of a LLO protein, thereby creating a recombinant nucleotide encoding an LLO-antigen fusion protein; and (b) administering the recombinant nucleotide or the LLO-antigen fusion to the subject; thereby inducing a CD8⁺ T cell-mediated immune response against a subdominant CD8⁺ T cell epitope of an antigen.

In one embodiment, provided herein is a method of increasing intratumoral ratio of CD8+/T regulatory cells, wherein and in another embodiment, the method comprises the step of administering to the subject a composition comprising the recombinant polypeptide, recombinant Listeria, or recombinant vector of the present invention.

In another embodiment, provided herein is a method of increasing intratumoral ratio of CD8+/T regulatory cells, wherein and in another embodiment, the method comprises the step of administering to the subject a composition comprising the recombinant polypeptide, recombinant Listeria, or recombinant vector of the present invention.

In another embodiment, provided herein is a method of delaying progression of spontaneous breast tumors in a subject, the method comprising the step of administering to the subject a therapeutically effective dose of the recombinant Listeria vaccine vector, wherein administering the recombinant Listeria induces epitope spreading to additional tumor associated antigens, for example those disclosed in U.S. Pat. Nos. 7,820,180, and 7,794,729.

In another embodiment, the immune response elicited by the methods and compositions provided herein comprises an immune response to at least one subdominant epitope of the antigen. In another embodiment, the immune response does not comprise an immune response to a subdominant epitope. In another embodiment, the immune response consists primarily of an immune response to at least one subdominant epitope. In another embodiment, the only measurable component of the immune response is an immune response to at least one subdominant epitope. Each type of immune response represents a separate embodiment of the present invention.

“Dominant CD8⁺ T cell epitope,” in one embodiment, refers to an epitope that is recognized by over 30% of the antigen-specific CD8⁺ T cells that are elicited by vaccination, infection, or a malignant growth with a protein or a pathogen or cancer cell containing the protein. In another embodiment, the term refers to an epitope recognized by over 35% of the antigen-specific CD8⁺ T cells that are elicited thereby. In another embodiment, the term refers to an epitope recognized by over 40% of the antigen-specific CD8⁺ T cells. In another embodiment, the term refers to an epitope recognized by over 45% of the antigen-specific CD8⁺ T cells. In another embodiment, the term refers to an epitope recognized by over 50% of the antigen-specific CD8⁺ T cells. In another embodiment, the term refers to an epitope recognized by over 55% of the antigen-specific CD8⁺ T cells. In another embodiment, the term refers to an epitope recognized by over 60% of the antigen-specific CD8⁺ T cells. In another embodiment, the term refers to an epitope recognized by over 65% of the antigen-specific CD8⁺ T cells. In another embodiment, the term refers to an epitope recognized by over 70% of the antigen-specific CD8⁺ T cells. In another embodiment, the term refers to an epitope recognized by over 75% of the antigen-specific CD8⁺ T cells. In another embodiment, the term refers to an epitope recognized by over 80% of the antigen-specific CD8⁺ T cells. In another embodiment, the term refers to an epitope recognized by over 85% of the antigen-specific CD8⁺ T cells. In another embodiment, the term refers to an epitope recognized by over 90% of the antigen-specific CD8⁺ T cells. In another embodiment, the term refers to an epitope recognized by over 95% of the antigen-specific CD8⁺ T cells. In another embodiment, the term refers to an epitope recognized by over 96% of the antigen-specific CD8⁺ T cells. In another embodiment, the term refers to an epitope recognized by over 97% of the antigen-specific CD8⁺ T cells. In another embodiment, the term refers to an epitope recognized by over 98% of the antigen-specific CD8⁺ T cells.

“Subdominant CD8⁺ T cell epitope,” in one embodiment, refers to an epitope recognized by fewer than 30% of the antigen-specific CD8⁺ T cells that are elicited by vaccination, infection, or a malignant growth with a protein or a pathogen or cancer cell containing the protein. In another embodiment, the term refers to an epitope recognized by fewer than 28% of the antigen-specific CD8⁺ T cells. In another embodiment, the term refers to an epitope recognized by over 26% of the antigen-specific CD8⁺ T cells. In another embodiment, the term refers to an epitope recognized by fewer than 24% of the antigen-specific CD8⁺ T cells. In another embodiment, the term refers to an epitope recognized by over 22% of the antigen-specific CD8⁺ T cells. In another embodiment, the term refers to an epitope recognized by fewer than 20% of the antigen-specific CD8⁺ T cells. In another embodiment, the term refers to an epitope recognized by over 18% of the antigen-specific CD8⁺ T cells. In another embodiment, the term refers to an epitope recognized by fewer than 16% of the antigen-specific CD8⁺ T cells. In another embodiment, the term refers to an epitope recognized by over 14% of the antigen-specific CD8⁺ T cells. In another embodiment, the term refers to an epitope recognized by over 12% of the antigen-specific CD8⁺ T cells. In another embodiment, the term refers to an epitope recognized by fewer than 10% of the antigen-specific CD8⁺ T cells. In another embodiment, the term refers to an epitope recognized by over 8% of the antigen-specific CD8⁺ T cells. In another embodiment, the term refers to an epitope recognized by fewer than 6% of the antigen-specific CD8⁺ T cells. In another embodiment, the term refers to an epitope recognized by fewer than 5% of the antigen-specific CD8⁺ T cells. In another embodiment, the term refers to an epitope recognized by over 4% of the antigen-specific CD8⁺ T cells. In another embodiment, the term refers to an epitope recognized by fewer than 3% of the antigen-specific CD8⁺ T cells. In another embodiment, the term refers to an epitope recognized by fewer than 2% of the antigen-specific CD8⁺ T cells. In another embodiment, the term refers to an epitope recognized by fewer than 1% of the antigen-specific CD8⁺ T cells. In another embodiment, the term refers to an epitope recognized by fewer than 0.5% of the antigen-specific CD8⁺ T cells.

Each type of the dominant epitope and subdominant epitope represents a separate embodiment of the present invention.

Methods of measuring immune responses are well known in the art, and include, e.g. measuring suppression of tumor growth, flow cytometry, target cell lysis assays (e.g. chromium release assay), the use of tetramers, and others. Each method represents a separate embodiment of the present invention.

The antigen in methods and compositions of the present invention is, in one embodiment, expressed at a detectable level on a non-tumor cell of the subject. In another embodiment, the antigen is expressed at a detectable level on at least a certain percentage (e.g. 0.01%, 0.03%, 0.1%, 0.3%, 1%, 2%, 3%, or 5%) of non-tumor cells of the subject. In another embodiment, the level of antigen expression is higher in a tumor cell when compared to a non-tumor cell (see FIG. 1). In one embodiment, “non-tumor cell” refers to a cell outside the body of the tumor. In another embodiment, “non-tumor cell” refers to a non-malignant cell. In another embodiment, “non-tumor cell” refers to a non-transformed cell. In another embodiment, the non-tumor cell is a somatic cell. In another embodiment, the non-tumor cell is a germ cell. Each possibility represents a separate embodiment of the present invention.

“Detectable level” refers, in one embodiment, to a level detectable by a standard assay. In one embodiment, the assay is an immunological assay. In one embodiment, the assay is enzyme-linked immunoassay (ELISA). In another embodiment, the assay is Western blot. In another embodiment, the assay is FACS. It is to be understood by a skilled artisan that any other assay available in the art can be used in the methods provided herein. In another embodiment, a detectable level is determined relative to the background level of a particular assay. Methods for performing each of these techniques are well known to those skilled in the art, and each technique represents a separate embodiment of the present invention.

In one embodiment, a treatment protocol of the present invention is therapeutic. In another embodiment, the protocol is prophylactic. In another embodiment, the vaccines of the present invention are used to protect people at risk for cancer such as breast cancer or other types of HER2-containing tumors because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. Following such treatments, the vaccines of the present invention are administered so that the CTL response to the tumor antigen of the vaccine destroys remaining metastases and prolongs remission from the cancer. In another embodiment, vaccines of the present invention are used to effect the growth of previously established tumors and to kill existing tumor cells. Each possibility represents a separate embodiment of the present invention.

In another embodiment, the nucleic acid molecule of methods and compositions of the present invention is operably linked to a promoter/regulatory sequence. In another embodiment, the first open reading frame of methods and compositions of the present invention is operably linked to a promoter/regulatory sequence. In another embodiment, the second open reading frame of methods and compositions of the present invention is operably linked to a promoter/regulatory sequence. In another embodiment, each of the open reading frames are operably linked to a promoter/regulatory sequence. Each possibility represents a separate embodiment of the present invention.

The skilled artisan, when equipped with the present disclosure and the methods provided herein, will readily understand that different transcriptional promoters, terminators, carrier vectors or specific gene sequences (e.g. those in commercially available cloning vectors) can be used successfully in methods and compositions of the present invention. As is contemplated in the present invention, these functionalities are provided in, for example, the commercially available vectors known as the pUC series. In another embodiment, non-essential DNA sequences (e.g. antibiotic resistance genes) are removed. Each possibility represents a separate embodiment of the present invention. In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art, and such methods are well known in the art, and are described in, for example, Sambrook et al. (1989, Molecular Cloning: A Laboratory Manual, Cold Spring Harbor Laboratory Press, New York) and Ausubei et al. (1997, Current Protocols in Molecular Biology, Green & Wiley, New York).

Antibiotic resistance genes are used in the conventional selection and cloning processes commonly employed in molecular biology and vaccine preparation. Antibiotic resistance genes contemplated in the present invention include, but are not limited to, gene products that confer resistance to ampicillin, penicillin, methicillin, streptomycin, erythromycin, kanamycin, tetracycline, cloramphenicol (CAT), neomycin, hygromycin, gentamicin and others well known in the art. Each gene represents a separate embodiment of the present invention.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, DC; Miller, 1992, A Short Course in Bacterial Genetics, Cold Spring Harbor Laboratory Press, Cold Spring Harbor, N.Y.) In another embodiment, the Listeria vaccine strain of the present invention is transformed by electroporation. Each method represents a separate embodiment of the present invention.

In another embodiment, conjugation is used to introduce genetic material and/or plasmids into bacteria. Methods for conjugation are well known in the art, and are described, for example, in Nikodinovic J et al. (A second generation snp-derived Escherichia coli-Streptomyces shuttle expression vector that is generally transferable by conjugation. Plasmid. 2006 Nov; 56(3):223-7) and Auchtung J M et al (Regulation of a Bacillus subtilis mobile genetic element by intercellular signaling and the global DNA damage response. Proc Natl Acad Sci USA. 2005 Aug 30; 102 (35):12554-9). Each method represents a separate embodiment of the present invention.

The term “Transforming,” in one embodiment, is used identically with the term “transfecting,” and refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the present invention.

Plasmids and other expression vectors useful in the present invention are described elsewhere herein, and can include such features as a promoter/regulatory sequence, an origin of replication for gram negative and gram positive bacteria, an isolated nucleic acid encoding a fusion protein and an isolated nucleic acid encoding an amino acid metabolism gene. Further, an isolated nucleic acid encoding a fusion protein will have a promoter suitable for driving expression of such an isolated nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of 5 bacteriophage lambda (PL and PR), the trp, recA, lacZ, lad, and gal promoters of E. coli, the alpha-amylase (Ulmanen et al, 1985. J. Bacteriol. 162:176-182) and the S28-specific promoters of B. subtilis (Gilman et al, 1984 Gene 32:11- 20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann Rev. Genet. 18:415-442). Further examples of promoter/regulatory elements contemplated in the present invention include, but are not limited to the Listeria1 prfA promoter, the Listeria1 hly promoter, the Listeria1 p60 promoter and the Listeria1 ActA promoter (GenBank Acc. No. NC_{—}003210) or fragments thereof.

In another embodiment, a plasmid of methods and compositions of the present invention comprises a gene encoding a fusion protein. In another embodiment, subsequences are cloned and the appropriate subsequences cleaved using appropriate restriction enzymes. The fragments are then, in another embodiment, ligated to produce the desired DNA sequence. In another embodiment, DNA encoding the antigen is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Thus, the gene for non-hemolytic LLO is PCR amplified, using a sense primer comprising a suitable restriction site and an antisense primer comprising another restriction site, e.g. a non-identical restriction site to facilitate cloning. The same is repeated for the isolated nucleic acid encoding an antigen. Ligation of the non-hemolytic LLO and antigen sequences and insertion into a plasmid or vector produces a vector encoding non-hemolytic LLO joined to a terminus of the antigen. The two molecules are joined either directly or by a short spacer introduced by the restriction site.

Each method represents a separate embodiment of the present invention.

The recombinant proteins of the present invention are synthesized, in another embodiment, using recombinant DNA methodology. This involves, in one embodiment, creating a DNA sequence that encodes the fusion protein, placing the DNA in an expression cassette, such as the plasmid of the present invention, under the control of a particular promoter/regulatory element, and expressing the protein. DNA encoding the fusion protein (e.g. non-hemolytic LLO/antigen) of the present invention is prepared, in another embodiment, by any suitable method, including, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-151); the diethylphosphoramidite method of Beaucage et al. (1981, Tetra. Lett., 22: 15 1859-1862); and the solid support method of U.S. Pat. No. 4,458,066.

In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences can be obtained by the ligation of shorter sequences. In another embodiment, subsequences are cloned and the appropriate subsequences cleaved using appropriate restriction enzymes. The fragments are then be ligated to produce the desired DNA sequence.

In another embodiment, the molecules of the fusion or recombinant protein provided herein are separated by a peptide spacer consisting of one or more amino acids, generally the spacer will have no specific biological activity other than to join the proteins or to preserve some minimum distance or other spatial relationship between them. In another embodiment, the constituent AA of the spacer are selected to influence some property of the molecule such as the folding, net charge, or hydrophobicity. In another embodiment, the nucleic acid sequences encoding the fusion or recombinant proteins are transformed into a variety of host cells, including E. coli, other bacterial hosts, such as Listeria, yeast, and various higher eukaryotic cells such as the COS, CHO and HeLa cells lines and myeloma cell lines. The recombinant fusion protein gene will be operably linked to appropriate expression control sequences for each host. Promoter/regulatory sequences are described in detail elsewhere herein. In another embodiment, the plasmid further comprises additional promoter regulatory elements, as well as a ribosome binding site and a transcription termination signal. For eukaryotic cells, the control sequences will include a promoter and an enhancer derived from e.g. immunoglobulin genes, SV40, cytomegalovirus, etc., and a polyadenylation sequence. In another embodiment, the sequences include splice donor and acceptor sequences.

In one embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

In one embodiment, provided herein is a method of administering the composition of the present invention. In another embodiment, provided herein is a method of administering the vaccine of the present invention. In another embodiment, provided herein is a method of administering the recombinant polypeptide or recombinant nucleotide of the present invention. In another embodiment, the step of administering the composition, vaccine, recombinant polypeptide or recombinant nucleotide of the present invention is performed with a recombinant form of Listeria comprising the composition, vaccine, recombinant nucleotide or expressing the recombinant polypeptide, each in its own discrete embodiment. In another embodiment, the administering is performed with a different bacterial vector. In another embodiment, the administering is performed with a DNA vaccine (e.g. a naked DNA vaccine). In another embodiment, administration of a recombinant polypeptide of the present invention is performed by producing the protein recombinantly, then administering the recombinant protein to a subject. Each possibility represents a separate embodiment of the present invention.

In one embodiment, the composition is administered to the cells of the subject ex vivo; in another embodiment, the composition is administered to the cells of a donor ex vivo; in another embodiment, the composition is administered to the cells of a donor in vivo, then is transferred to the subject. Each possibility represents a separate embodiment of the present invention.

In one embodiment, a vaccine or immunogenic composition of the present invention is administered alone to a subject. In another embodiment, the vaccine or immunogenic composition is administered together with another cancer therapy. Each possibility represents a separate embodiment of the present invention.

The terms “contacting” or “administering,” in one embodiment, refer to directly contacting the cancer cell or tumor with a composition of the present invention. In another embodiment, the terms refer to indirectly contacting the cancer cell or tumor with a composition of the present invention. In another embodiment, methods of the present invention include methods in which the subject is contacted with a composition of the present invention after which the composition is brought in contact with the cancer cell or tumor by diffusion or any other active transport or passive transport process known in the art by which compounds circulate within the body. Each possibility represents a separate embodiment of the present invention.

Various embodiments of dosage ranges are contemplated by this invention. In one embodiment, in the case of vaccine vectors, the dosage is in the range of 0.4 LD₅₀/dose. In another embodiment, the dosage is from about 0.4-4.9 LD₅₀/dose. In another embodiment the dosage is from about 0.5-0.59 LD₅₀/dose. In another embodiment the dosage is from about 0.6-0.69 LD₅₀/dose. In another embodiment the dosage is from about 0.7-0.79 LD₅₀/dose. In another embodiment the dosage is about 0.8 LD₅₀/dose. In another embodiment, the dosage is 0.4 LD₅₀/dose to 0.8 of the LD₅₀/dose.

In another embodiment, the dosage is 10⁷ bacteria/dose. In another embodiment, the dosage is 1.5×10⁷ bacteria/dose. In another embodiment, the dosage is 2×10⁷ bacteria/dose. In another embodiment, the dosage is 3×10⁷ bacteria/dose. In another embodiment, the dosage is 4×10⁷ bacteria/dose. In another embodiment, the dosage is 6×10⁷ bacteria/dose. In another embodiment, the dosage is 8×10⁷ bacteria/dose. In another embodiment, the dosage is 1×10⁸ bacteria/dose. In another embodiment, the dosage is 1.5×10⁸ bacteria/dose. In another embodiment, the dosage is 2×10⁸ bacteria/dose. In another embodiment, the dosage is 3×10⁸ bacteria/dose. In another embodiment, the dosage is 4×10⁸ bacteria/dose. In another embodiment, the dosage is 6×10⁸ bacteria/dose. In another embodiment, the dosage is 8×10⁸ bacteria/dose. In another embodiment, the dosage is 1×10⁹ bacteria/dose. In another embodiment, the dosage is 1.5×10⁹ bacteria/dose. In another embodiment, the dosage is 2×10⁹ bacteria/dose. In another embodiment, the dosage is 3×10⁹ bacteria/dose. In another embodiment, the dosage is 5×10⁹ bacteria/dose. In another embodiment, the dosage is 6×10⁹ bacteria/dose. In another embodiment, the dosage is 8×10⁹ bacteria/dose. In another embodiment, the dosage is 1×10¹⁰ bacteria/dose. In another embodiment, the dosage is 1.5×10¹⁰ bacteria/dose. In another embodiment, the dosage is 2×10¹⁰ bacteria/dose. In another embodiment, the dosage is 3×10¹⁰ bacteria/dose. In another embodiment, the dosage is 5×10¹⁰ bacteria/dose. In another embodiment, the dosage is 6×10¹⁰ bacteria/dose. In another embodiment, the dosage is 8×10¹⁰ bacteria/dose. In another embodiment, the dosage is 8×10⁹ bacteria/dose. In another embodiment, the dosage is 1×10¹¹ bacteria/dose. In another embodiment, the dosage is 1.5×10¹¹ bacteria/dose. In another embodiment, the dosage is 2×10¹¹ bacteria/dose. In another embodiment, the dosage is 3×10¹¹ bacteria/dose. In another embodiment, the dosage is 5×10¹¹ bacteria/dose. In another embodiment, the dosage is 6×10¹¹ bacteria/dose. In another embodiment, the dosage is 8×10¹¹ bacteria/dose. Each possibility represents a separate embodiment of the present invention.

[000109] In one embodiment, a vaccine or immunogenic composition of the present invention is administered alone to a subject. In another embodiment, the vaccine or immunogenic composition is administered together with another cancer therapy. Each possibility represents a separate embodiment of the present invention.

In one embodiment, the construct or nucleic acid molecule is integrated into the Listeria1 chromosome using homologous recombination. Techniques for homologous recombination are well known in the art, and are described, for example, in Baloglu S, Boyle S M, et al (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In this case, a recombinant LM strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants. Each technique represents a separate embodiment of the present invention.

In another embodiment, the construct or nucleic acid molecule is integrated into the Listeria1 chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule provided herein is integrated into the Listeria1 chromosome using phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment of the present invention.

In another embodiment, one of various promoters is used to express the antigen or fusion protein containing same. In one embodiment, an LM promoter is used, e.g. promoters for the genes hly, actA, pica, plcB and mpl, which encode the Listeria1 proteins hemolysin, actA, phosphotidylinositol-specific phospholipase, phospholipase C, and metalloprotease, respectively. Each possibility represents a separate embodiment of the present invention.

In another embodiment, the term “homology,” when in reference to any nucleic acid sequence provided herein similarly indicates a percentage of nucleotides in a candidate sequence that are identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 1-8 of greater than 70%. In another embodiment, “homology” refers to identity to a sequence selected from SEQ ID No: 1-8 of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7. 6), 5× Denhardt's solution, 10% dextran sulfate, and 20 μg/ml denatured, sheared salmon sperm DNA.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

In another embodiment, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals or organisms. This can be readily carried out by using hybridization probes to identify the same genetic locus in a variety of individuals or organisms. Any and all such nucleotide variations and resulting amino acid polymorphisms or variations that are the result of natural allelic variation and that do not alter the functional activity are intended to be within the scope of the invention.

### Pharmaceutical Compositions

The pharmaceutical compositions containing vaccines and compositions of the present invention are, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of the methods and compositions provided herein, the vaccines or compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the vaccines or compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods:

### Mice

Balb/c female mice (6-8 week old) from Charles River Laboratories were utilized for all experiments involving the 4T1 tumor line. FVB/NJ female mice (6-8 week old) from Jackson Laboratories were utilized for all experiments involving the NT2 tumor line. A rat Her2/neu transgenic mouse strain in the FVB/NJ background was utilized in studies involving spontaneous tumor formation and for prevention studies of autochthonous mammary tumor formation was housed and bred at the animal core facility at the University of Pennsylvania. All mouse experiments were performed in accordance with the regulations of the Institutional Animal Care and Use Committee of the University of Pennsylvania.

### Listeria Strains

To construct an attenuated Listeria-based ISG15 vaccine, first the gene encoding murine ISG15 was amplified from a construct containing murine ISG15 cDNA from Balb/c mice with the following primers: Lm-LLO-ISG15.FOR 5′-TAAT-CTCGAG-ATGGCCTGGGACCTAAAG-3′ (SEQ ID NO: 9) and Lm-LLO-ISG15.REV 5′-ATTA-ACTAGT-TTAGGCACACTGGTCCCC-3′ (SEQ ID NO: 10). The XhoI sequence underlined in the forward primer and the SpeI sequence underlined in the reverse primer were utilized for ligation. Each fragment amplicon was restriction-enzyme digested and ligated into the Listeria expression plasmid, pGG34. Each sequence was genetically fused downstream to the sequence encoding truncated Listeriolysin O (tLLO) under the control of the hly promoter. Subsequently, pGG34-LLO-IS G15 was electroporated into the attenuated Listeria monocytogenes (Lm) strain, XFL7, and plasmid containing colonies were selected for resistance on BHI-chloramphenicol plates. To confirm proper construction of Lm-LLO-ISG15, the attenuated Listeria-based vaccine was grown in BHI-chloramphenicol selection media and secreted proteins were precipitated with trichloroacetic acid. After boiling in SDS sample buffer, secreted proteins were subject to SDS-PAGE analysis and transferred to a PVDF membrane. Western analysis on the membrane was performed with anti-mouse ISG15 antibody (Santa Cruz Biotech, Santa Cruz, Calif.) to confirm secretion of the tLLO-ISG15 fusion protein, anti-chicken ovalbumin with 3A11.2 monoclonal antibody and wild-type LLO with B3-19 monoclonal antibody. The control vaccine, Lm-LLO-OVA, consisting of tLLO genetically fused to chicken ovalbumin was similarly constructed. All Listeria-based vaccines were administered intraperitoneally (i.p.) at either 2×10⁸ or 5×10⁸ CFU in 200 μl of PBS. The control vaccines Lm-LLO-OVA and Lm-LLO-NYESO-1 were similarly constructed.

Cell lines

The metastatic breast cancer tumor line 4T1 was utilized in tumor implantation studies in Balb/c mice. The NT2 breast cancer cell line that overexpresses rat Her2/neu was utilized for tumor implantation studies in FVB mice. 4T1-Luc was maintained in DMEM supplemented with 10% fetal calf serum, 2 mM _{L}-glutamine, 1 mM sodium pyruvate, 50 U/mL penicillin, and 50 μg/mL streptomycin. NT2 cells were maintained in RPMI 1640 medium supplemented with 10% fetal calf serum, 20 μg/mL insulin, 2 mM _{L}-glutamine, 1 mM sodium pyruvate, 50 U/mL penicillin, and 50 μg/mL streptomycin. The non-transformed NIH-3T3 fibroblast cell line obtained from ATCC. NIH-3T3 cells were maintained in DMEM supplemented with 10% fetal calf serum, 2 mM _{L}-glutamine, 1 mM sodium pyruvate, 50 U/mL penicillin, and 50 μg/mL streptomycin.

### ISG15 Expression in Normal and Tumor Murine Tissue

RNA was extracted from tissue or cells using the RNeasy RNA extraction kit from Qiagen and converted to cDNA. The cDNA was then subjected to qPCR analysis with primers specific for ISG15 qISG15.FOR 5′-ATGGCCTGGGACCTAAAG-3′ (SEQ ID NO: 11) and qISG15.REV 5′-TTAGGCACACTGGTCCCC-3′ (SEQ ID NO: 12), 18S rRNA 18SRNA.FOR 5′-CGGCTACCACATCCAAGGAA-3′ (SEQ ID NO: 13) and 18SRNA.REV 5′-GCTGGAATTACCGCGGCT-3′ (SEQ ID NO: 14), and β-actin ACTIN.FOR 5′-GTGGGCCGCTCTAGGCACCAA-3′ (SEQ ID NO: 15) and ACTIN.REV 5′-CTCTTTGATGTCACGCACGATTTC-3′ (SEQ ID NO: 16). ISG15 expression was normalized to either 18S rRNA (FIG. 1C and D) or β-actin (FIG. 1A).

### Western Blot Analysis of Mammary Tissue Lysates

Normal mammary tissue from FVB/N mice (n=4) and autochthonous mammary tumor tissue from HER2/neu transgenic mice in the FVB/N background (n=9) were excised and processed into lysates. Briefly, tissue samples were snap-frozen in liquid N₂, pulverized, and solubilized in lysis buffer (PBS with 2% Triton X-100 and 0.02% saponin) supplemented with protease inhibitor cocktail. Lysates were mixed with 4×LDS Sample Loading Buffer and subjected to SDS-PAGE. After transfer of separated proteins to a PVDF membrane, western blot analysis was performed with anti-mouse ISG15 antibody. Separately, the same lysates were subjected to SDS-PAGE and the gel stained with Coomassie stain to visualize total proteins as a measure of protein loading.

Tumor Immunotherapy with ISG15 Peptides.

4T1-Luc tumor cells (10⁵) were implanted into the mammary tissue of Balb/c mice and mice were subsequently vaccinated on day 5, 12, and 19 with either 100 μl of PBS or 50 μg CpG oligodeoxynucleotides (ODN) mixed with control, HIV-gag H-2K^{d} CTL epitope peptide (AMQMLKETI) (SEQ ID NO: 17), or ISG15-specific peptides (100 μg), pISG15 d1(RGHSNIYEV) (SEQ ID NO: 18) and pISG15 d2(LGPSSTVML) (SEQ ID NO: 19), in 100 μL of PBS s.c. proximal to the cervical lymph nodes. Tumor volume was monitored by perpendicular caliper measurements throughout the course of the experiment. Tumor volume was calculated as (tumor diameter)³/2.

### ISG15 Peptide Tumor Load Study

4T1-Luc tumor cells (10⁵) were implanted into the mammary tissue and mice were subsequently vaccinated on day 4, 11, and 18 with either 50 ug of CpG alone in 100 ul of PBS or CpG (50 ug) along with control or ISG15-specific peptides (100 ug) in 100 ul of PBS subcutaneously proximal to the cervical lymph nodes. At experimental end on day 32, tumor mass of each vaccinated group was measured, tumors were analyzed for ISG15-specific IFN-γ responses as described in ELISpot Analysis and lung metastates measured as described in Metastatic Tumor Study.

### Metastatic Tumor Study

4T1-Luc tumor cells (10⁵) were implanted into the mammary tissue and mice were subsequently vaccinated on day 4, 11, and 18 with either peptide or Listeria-based vaccines. Mice were then sacrificed on day 32 and lungs isolated and perfused with PBS. Lung surface metastatic nodules per lung were then counted with a Nikon SMZ1B Zoom Stereomicroscope attached to a Fostec 8375 Illuminator and Ringlight.

### ELISpot Analysis

The 96-well filtration plates (Millipore, Bedford, Mass.) were coated with 15 μg/ml rat anti-mouse IFN-γ antibody in 100 μl of PBS. After overnight incubation at 4° C., the wells were washed and blocked with DMEM supplemented with 10% fetal calf serum. For FIG. 2C, splenocytes from each experimental group were added to the wells along with HIV-gag H-2K^{d} CTL epitope peptide (AMQMLKETI) (SEQ ID NO: 20) or predicted ISG15-specific H-2K^{d} CTL epitope peptides, ISG15-d1(RGHSNIYEV) (SEQ ID NO: 21) and ISG15-d2(LGPSSTVML) (SEQ ID NO: 22) (5 μg/ml ) plus IL-2 (5 U/ml). ISG15-specific H-2K^{d} CTL epitope were predicted from the ISG15 protein sequence in Balb/c mice using RANKPEP prediction software at http://bio.dfci.harvard.edu/Tools/rankpep.html. For FIG. 5B, splenocytes from each experimental group were added to the wells along with HIV-gag H-2K^{d} CTL epitope peptide (AMQMLKETI) (SEQ ID NO: 23) or Her2/neu-specific H-2K^{d} epitope peptides Her2-EC1 (PYNYLSTEV) (SEQ ID NO: 24), Her2-EC2 (LFRNPHQALL) (SEQ ID NO: 25), and Her2-IC1 (PYVSRLLGI) (SEQ ID NO: 26). Cells were incubated at 37° C. for 24 h. The plate was washed followed by incubation with 1 μg/ml biotinylated IFN-γ antibody (clone R4-6A2, MABTECH, Mariemont, Ohio) in 100 μl PBS at 4° C. overnight. After washing, 1:100 streptavidin-horseradish peroxidase in 100 μl PBS were added and incubated for 1 hr at room temperature. Spots were developed by adding 100 μl of substrate after washing and incubated at room temperature for 15 min Color development was stopped by washing extensively in dH₂O and spot-forming cells (SFC) were counted with an ELISpot reader.

### Depletion Experiment

CD8⁺ cells were depleted in 4T1-Luc tumor-bearing mice by injecting the mice with 0.5 mg of α-CD8 antibody (monoclonal antibody clone 2.43) on days 6, 7, 8, 10, 12, and 14 post-tumor implantation. A control group of mice were also treated under the same conditions but with an isotype matched, control antibody specific for beta-galactosidase. The concurrent tumor load study was adhered to as described in “Tumor immunotherapy with Lm-LLO-ISG15” in this methods section.

### Winn Assay for In Vivo Determination of Effector Cell.

The Winn assay was performed as previously described with some modification. Briefly, 4T1-Luc tumor cells (2×10⁵) mixed with CD4-depleted splenocytes (depletion with CD4⁺ Dynabeads and confirmed by FACS analysis) from either twice control Lm vaccinated or twice Lm-LLO-ISG15 vaccinated Balb/c mice (2×10⁷) at a ratio of 1 tumor cell to 100 CD4-depleted splenocytes were implanted in the mammary tissue. Tumor development was then measured as described in “Tumor immunotherapy with Lm-LLO-ISG15” in this methods section.

### Detection of HER2/Neu-Specific Tumor Infiltrating Lymphocytes (TILS)

Balb/c mice were implanted with 4T1-Luc tumors and immunized i.p. with control Lm or Lm-LLO-ISG15 and boosted 7 days later. Tumors were harvested 9 days after boosting and manually dissociated into a single-cell suspension. The tumor cell suspension was then Ficoll-purified to remove dead cells and cellular debris by excluding the low-density fraction after centrifugation. The remaining tumor cells were then subjected to three-color flow cytometry for CD8 (53-6.7, FITC conjugated), CD62 ligand (CD62L; MEL-14, APC conjugated), and HER2/neu-EC2 H-2D^{q} tetramer-PE conjugated (specific for PDSLRDLSVF) using a FACSCalibur flow cytometer with CellQuest software. Tetramers were provided by the National Institute of Allergy and Infectious Diseases Tetramer Core Facility and used at a 1/200 dilution. Results were analyzed as described above to compare the ability of Lm-LLO-ISG15 to induce tetramer⁺, CD8⁺, CD62L⁻, Her2/neu-specific TILs in comparison to control Lm vaccination.

### Statistical Analyses

One-tailed student's t-tests were performed for all final tumor volume, metastatic load and immune response studies with Welch's correction applied for gene expression studies with autochthonous HER2/neu mammary tumors. Log rank test was performed for autochthonous HER2/neu mammary tumor incidence studies. Statistical analyses were performed using GraphPad Prism version 4.0a for Macintosh (www.graphpad.com). Significant p-values for all comparisons are depicted in figures as follows: *=p-value<0.05, **=p-value<0.01, and ***=p-value<0.001.

### Results

### Example 1

### Elevated Expression of ISG15 in Murine Breast Tumors

The elevated expression of ISG15 in human malignancies is well-characterized in numerous tumor models. However, there is a lack of evidence for similar increased levels of ISG15 in murine tumor models. To determine if ISG15 expression is elevated in a murine model for breast cancer, ISG15 expression was assayed in autochthonous mouse mammary tumors from HER2/neu transgenic mice, mouse mammary tumor cell lines and a panel of normal and non-transformed mammary tissues and cell lines. As observed in human breast cancer, expression of ISG15 mRNA is significantly elevated in the autochthonous mouse mammary tumors in comparison to normal mouse mammary tissue (FIG. 1A). To confirm the elevated ISG15 mRNA expression results in elevated protein production, Western blot analysis with anti-ISG15 antibody was performed with lysates of normal and HER2/neu tumor mouse mammary tissue. In comparison to normal mouse mammary tissue (FIG. 1B, top panel, lane 1), the conjugated form of ISG15 protein (bands above 20 kD marker) is elevated in HER2/neu mammary tumor tissue (FIG. 1B, top panel, lanes 2-5). Elevated expression of the unconjugated form of ISG15 protein is also evident in mouse mammary tumor tissue (FIG. 1B, top panel, lanes 2 and 4) in comparison to normal mammary tissue (FIG. 1B, top panel, lane 1). Equivalent protein loading is evident by probing for expression of the housekeeping protein, GAPDH, with the same lysates (FIG. 1B, bottom panel, lanes 1-5). ISG15 mRNA expression was similarly elevated in mouse mammary tumor cell lines, 4T1-Luc and NT2, in comparison to normal mouse mammary tissue and a non-transformed mouse cell line, NIH-3T3 (FIG. 1C). To alleviate concerns of elevated ISG15 expression in non-malignant tissues, ISG15 mRNA expression was analyzed in a panel of normal mouse tissues in comparison to HER2/neu mammary mouse tumor tissue. Significantly elevated expression of ISG15 mRNA in mammary tumor tissue was similarly observed when compared against each normal tissue type (FIG. 1D). This expression analysis confirms that ISG15 expression is significantly elevated in mouse models of breast cancer. Together with the finding that ISG15 mRNA is nominally expressed in a panel of normal tissues, this suggests that ISG15 may be a promising novel tumor-associated antigen (TAA).

### Example 2

### Construction of an ISG15-Specific CTL Vaccine

To assess the potential for ISG15 as a novel TAA, a Listeria-based CTL vaccine was developed to target tumors with elevated ISG15 expression. Construction of the vaccine, Lm-LLO-ISG15, was accomplished by genetically fusing the mouse ISG15 gene from Balb/c mice downstream of the gene encoding a truncated form of Listeriolysin O (tLLO), already present in the Listeria monocytogenes (Lm) expression vector pGG34, which contains a signal sequence to allow for proper secretion of the fusion protein. The pGG34-LLO-ISG15 construct was subsequently electroporated into the attenuated competent Lm strain, XFL7 (FIG. 2A). Proper secretion of the tLLO-ISG15 fusion protein was confirmed by Western blot analysis with anti-mouse ISG15 antibody against TCA-precipitated proteins from the media of an Lm-LLO-ISG15 growth culture (FIG. 2B, top panel). Similar production and secretion of a fusion protein of tLLO fused to chicken ovalbumin was observed from our control Lm when probed with anti-ovalbumin antibody (FIG. 2B, middle panel). Secreted proteins from Lm-LLO-ISG15 and the control Lm were also probed with wild-type LLO antibody to confirm equivalent secreted protein loading (FIG. 2B, bottom panel). Generation of ISG15-specific CTL responses was assayed by administering both Lm-LLO-ISG15 and a control Lm vaccine to female Balb/c mice, weekly, starting at week 6. One week after the third vaccination, splenocytes from each vaccination group were subjected to ELISpot analysis to investigate IFN-γ responses against a control epitope and two ISG15-specific H2-K^{d}-restricted CD8+ T-cell epitopes predicted by RANKPEP. A significant increase in IFN-γ secreting SFCs was observed only in the splenocytes from the Lm-LLO-ISG15 vaccinated mice after stimulation with each predicted ISG15-specific CTL epitope in comparison to control peptide stimulation (FIG. 2C). These results suggest that an ISG15-specific adaptive response can be generated by an attenuated Lm-based CTL vaccine against ISG15.

While under normal conditions, ISG15 expression is at low or undetectable levels in normal tissues, however, there is evidence for elevated ISG15 expression at the placental implantation site during pregnancy. To determine if an ISG15-specific immune response may severely impact fertility in Lm-LLO-ISG15 vaccinated female mice, a pregnancy study was performed. In comparison to control Lm vaccinated female mice, the fertility of Lm-LLO-ISG15 vaccinated female mice was not significantly impaired as measured by litter size and pup weight (FIGS. 2D and E, respectively). Generation of an ISG15-specific adaptive immune response with no obvious adverse effects encouraged examination of its efficacy in mouse models for breast cancer.

### Example 3

### Therapeutic Impact on Murine Breast Tumors after Lm-LLO-ISG15 Vaccination

The therapeutic potential of an ISG15-specific adaptive immune response generated by Lm-LLO-ISG15 against breast cancer was initially investigated against implanted primary and metastatic mouse models of breast cancer. Implantation of NT2 tumor cells s.c. in the hind flank of FVB/N mice and subsequent vaccination with Lm-LLO-ISG15 resulted in significantly reduced tumor volume as compared to control vaccination (FIG. 3A). Similarly, Lm-LLO-ISG15 therapeutic vaccination significantly inhibited the growth of mammary tissue-implanted 4T1-Luc primary tumors (FIG. 3B). The ability of 4T1-Luc tumors to naturally metastasize after implantation in the mammary gland (29-30) allowed further investigation into the efficacy of an ISG15-specific CTL response against a more aggressive model for breast cancer. Significant reductions in the appearance of 4T1-Luc metastatic lung lesions were observed after Lm-LLO-ISG15 administration in comparison to control Lm (FIG. 3C).

### Example 4

### Delayed Progress of HER2/Neu+ Autochthonous Mammary Tumors and Epitiope Spreading by Lm-LLO-ISG15

To determine if Lm-LLO-ISG15 could also provide therapeutic efficacy in a more clinically relevant model of human breast tumor development, we utilized a FVB/N HER2/neu transgenic mouse model that, in the absence of therapeutic intervention, develops autochthonous mammary tumors past 4 months of age. Transgenic female mice were vaccinated every three weeks with Lm-LLO-ISG15 or a control Lm from week 6 to 21 after birth and subsequently monitored for mammary tumor incidence. Mice administered Lm-LLO-ISG15 demonstrated a significant delay to tumor progression in comparison to a control Lm vaccinated group (p<0.0001) (FIG. 4A). In fact, greater than 80 percent of Lm-LLO-ISG15 vaccinated mice are still tumor-free by week 49 after birth while all control Lm vaccinated mice have developed mammary tumors with a median time to progression of 31 weeks. To determine if the infiltration of ISG15-specific CTLs into autochthonous tumors after Lm-LLO-ISG15 vaccination could be a possible mechanism for this delayed progression, an IFNγ ELISpot analysis was performed on TILs of these tumors after Lm-LLO-ISG15 vaccination. After allowing for autochthonous tumors to form, tumor-bearing mice were vaccinated twice on day 0 and 7 with either a Control Lm vaccine or Lm-LLO-ISG15. One week after the last vaccination, tumors were excised and TILs purified and processed for ELISpot analysis. As expected, the tumors of Lm-LLO-ISG15 vaccinated contain a significantly greater number of TILs specific for ISG15, as measured by their ability to secrete IFNγ after ISG15 epitope peptide stimulation, than the tumors of Control Lm vaccinated mice (FIG. 4B). These results suggest that the delayed progression of autochthonous mammary tumors by Lm-LLO-ISG15 is, in part, mediated by infiltration of ISG15-specific CTLs.

Recent studies demonstrate that the clinical efficacy of cancer vaccines significantly correlates with their ability to stimulate cross-priming and epitope spreading to additional TAAs. Similar results were observed previously using Lm-based cancer vaccines where development of epitope spreading to additional TAAs was associated with vaccine efficacy. To assess whether epitope spreading is developing after Lm-LLO-ISG15 vaccination, an ELISpot to detect HER2/neu-specific responses was performed with splenocytes from NT2 tumor-bearing mice after administration of either control Lm or Lm-LLO-ISG15. Splenocytes of Lm-LLO-ISG15 vaccinated mice contained significantly greater numbers of SFCs specific for known CTL epitopes within HER2/neu compared to control Lm vaccinated mice (FIG. 4C). This result suggests that Lm-LLO-ISG15 vaccination results in epitope spreading to additional TAAs. In fact, evidence for epitope spreading was also observed after Lm-LLO-ISG15 vaccination against 4T1-Luc tumors, a tumor cell line that expresses Her2/neu very weakly. 4T1-Luc tumors from Lm-LLO-ISG15 vaccinated mice contained a significantly higher percentage of Her2/neu-specific CD8⁺ 62L ^{- TILs than} 4T1-Luc tumors from control Lm vaccinated mice (FIG. 4D). While epitope spreading to HER2/neu may provide some therapeutic efficacy, it is unclear if this secondary response is robust enough to warrant cardiotoxicity safety concerns. In summary, these tumor load studies demonstrate that vaccination against ISG15 can inhibit the growth of primary implanted mouse mammary tumors, inhibit metastatic spread, delay progression of autochthonous mammary tumors and generate epitope spreading to additional TAAs.

### Example 5

### Therapeutic Impact of ISG15 Vaccination is CD-8 Dependent

While the generation of robust IFN-γ responses and significant therapeutic tumor impact are suggestive of strong CTL responses, the dependence of ISG15-specific CD8+ T cell function in Lm-LLO-ISG15 efficacy was investigated. Depletion of CD8⁺ cells in 4T1-Luc tumor-bearing mice completely abrogates the anti-tumor efficacy of Lm-LLO-ISG15 compared to mock depletion with a control antibody (FIG. 5A). As an in vivo measure of ISG15-specific CTL tumor cell lysis, we performed a Winn assay to assess whether splenocytes enriched for CD8⁺ T cells from Lm-LLO-ISG15 vaccinated mice could directly inhibit 4T1-Luc tumor formation. Splenocytes from mice twice-vaccinated with either Lm-LLO-ISG15 or a control Lm were depleted of CD4⁺ cells and incubated briefly with 4T1-Luc tumor cells. The tumor cell and splenocyte mixture was then implanted into the mammary tissue of Balb/c mice and tumor progression monitored. CD8⁺ T-cell-enriched splenocytes from Lm-LLO-ISG15 vaccinated mice significantly inhibited tumor growth in comparison to those from control Lm vaccinated mice (FIG. 5B). Additionally, all control Lm splenocyte-receiving mice developed tumors by day 21 post-implantation while 40% of mice receiving ISG15-specific splenocytes were still tumor-free at day 43 (FIG. 5C). This result suggests that Lm-LLO-ISG15 induces a CD8-dependent adaptive immune response that results in direct lysis of tumor cells and is likely mediated by CD8⁺ T cells.

### Example 6

### Expansion of ISG15-Specific CTL Clones In Vivo Results in Anti-Tumor Responses

To assess whether expansion of a single ISG15-specific CD8⁺ T cell clone can result in anti-tumor efficacy, mice were implanted with 4T1-Luc tumor cells and vaccinated with either PBS alone or an adjuvant, CpG ODN, mixed with each ISG15 H2K^{d} epitope peptide or a control peptide. In mice vaccinated with CpG ODN and ISG15 H2K^{d} peptides, 4T1-Luc tumor volume and tumor mass were significantly reduced in comparison to PBS alone and control peptide vaccination (FIGS. 6A and B, respectively). 4T1-Luc tumor lung metastases were also significantly reduced after vaccination with each ISG15 peptide in comparison to PBS alone or control peptide vaccination (FIG. 6C). Additionally, IFNγ secretion in response to stimulation with each ISG15 H2K^{d} epitope peptides was observed in TILs only from mice that were vaccinated with their respective ISG15 H2K^{d} epitope peptide suggesting that there was a successful expansion ISG15-specfic CTLs that trafficked to the targeted tumor (FIGS. 6D and E). These data strongly suggest that expansion of ISG15-specific CD8⁺ T cells can directly inhibit growth of tumors with elevated expression of ISG15.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A recombinant Listeria vaccine vector comprising a recombinant nucleic acid encoding a recombinant polypeptide, wherein said recombinant polypeptide comprises a non-hemolytic N-terminal Listeriolysin (LLO) fused to a tumor antigen, and wherein said tumor antigen is ISG15.

2. A recombinant nucleic acid molecule encoding the fusion polypeptide of claim 1.

3. A recombinant polypeptide encoded by the recombinant nucleic acid molecule of claim 2.

4. The recombinant Listeria of claim 1, wherein said Listeria strain is Listeria monocytogenes.

5. A method of diagnosing a tumor growth in a subject, the method comprising the step of obtaining a biological sample from the subject and measuring the expression profile of an ISG15 antigen in the biological sample, wherein when the ISG15 expression level is observed to be elevated in the subject over the levels observed in that of a control sample the subject is effectively diagnosed as having a ISG15-expressing tumor growth.

6. The method of claim 5, wherein said biological sample is tissue, blood, urine, semen, sputa, spinal chord fluid.

7. The method of claim 5, wherein said tumor growth is a cancer.

8. The method of claim 7, wherein said cancer is breast cancer, cervical cancer, bladder cancer, oral squamous carcinoma, melanoma, prostate cancer, endometrial cancer or a combination thereof.

9. A method of enhancing an anti-ISG15 immune response in a subject, said method comprising the step of administering to said subject a therapeutically effective dose of said recombinant Listeria of claim 1.

10. The method of claim 9, wherein said administering is via injection.

11. A method of eliciting an anti-ISG15 adaptive immune response in a subject, said method comprising the step of administering to said subject a therapeutically effective dose of the composition of claim 1.

12. The method of claim 11, wherein said administe is via injection.

13. A method of treating a tumor growth in a subject, said method comprising the step of administering to said subject a therapeutically effective dose of said recombinant Listeria of claim 1.

14. The method of claim 14, wherein said tumor growth is a cancer.

15. The method of claim 15, wherein said cancer is breast cancer, bladder cancer, oral squamous carcinoma, melanoma, prostate cancer, endometrial cancer or a combination thereof.

16. The method of claim 14, wherein said administering is via injection.

17. A method of treating a ISG15 antigen-expressing tumor growth in a subject, said method comprising the step of administering to said subject a therapeutically effective dose of said recombinant Listeria of claim 1.

18. The method of claim 18, wherein said tumor growth is a cancer.

19. The method of claim 19, wherein said cancer is breast cancer, bladder cancer, oral squamous carcinoma, melanoma, prostate cancer, endometrial cancer or a combination thereof.

20. The method of claim 18, wherein said administering is via injection.

21. A method of treating a Her-2/neu antigen-expressing tumor growth in a subject, said method comprising the step of administering to said subject a therapeutically effective dose of said recombinant Listeria of claim 1.

22. The method of claim 22, wherein said tumor growth is a cancer.

23. The method of claim 23, wherein said cancer is breast cancer, bladder cancer, oral squamous carcinoma, melanoma, prostate cancer, endometrial cancer or a combination thereof.

24. The method of claim 22, wherein said administering is via injection.

25. A method of treating a metastases in a subject, said method comprising the step of administering to said subject a therapeutically effective dose of said recombinant Listeria of claim 1.

26. The method of claim 26, wherein said tumor growth is a cancer.

27. The method of claim 27, wherein said cancer is breast cancer, bladder cancer, oral squamous carcinoma, melanoma, prostate cancer, endometrial cancer or a combination thereof.

28. The method of claim 28, wherein said administering is via injection.

29. A method of preventing the onset of a tumor in a subject, said method comprising the step of administering to said subject a therapeutically effective dose of said recombinant Listeria of claim 1.

30. The method of claim 30, wherein said tumor growth is a cancer.

31. The method of claim 31, wherein said cancer is breast cancer, bladder cancer, oral squamous carcinoma, melanoma, prostate cancer, endometrial cancer or a combination thereof.

32. The method of claim 30, wherein said administering is via injection.

33. A method of preventing the onset of a ISG15 antigen-expressing tumor in a subject, said method comprising the step of administering to said subject a therapeutically effective dose of said recombinant Listeria of claim 1.

34. The method of claim 34, wherein said tumor growth is a cancer.

35. The method of claim 35, wherein said cancer is breast cancer, bladder cancer, oral squamous carcinoma, melanoma, prostate cancer, endometrial cancer or a combination thereof.

36. The method of claim 20, wherein said administering is via injection.

37. A method of preventing the onset of a Her2/neu antigen-expressing tumor in a subject, said method comprising the step of administering to said subject a therapeutically effective dose of said recombinant Listeria of claim 1.

38. The method of claim 38, wherein said tumor growth is a cancer.

39. The method of claim 39, wherein said cancer is breast cancer, bladder cancer, oral squamous carcinoma, melanoma, prostate cancer, endometrial cancer or a combination thereof.

40. The method of claim 38, wherein said administering is via injection.

41. A method of preventing the onset of a Her2/neu antigen-expressing tumor in a subject, said method comprising the step of administering to said subject a therapeutically effective dose of said recombinant Listeria of claim 1.

42. The method of claim 42, wherein said tumor growth is a cancer.

43. The method of claim 43, wherein said cancer is breast cancer, bladder cancer, oral squamous carcinoma, melanoma, prostate cancer, endometrial cancer or a combination thereof.

44. The method of claim 42, wherein said administering is via injection.

45. A method of preventing metastatic tumor growth in a subject, said method comprising the step of administering to said subject a therapeutically effective dose of said recombinant Listeria of claim 1.

46. The method of claim 46, wherein said tumor growth is a cancer.

47. The method of claim 47, wherein said cancer is breast cancer, bladder cancer, oral squamous carcinoma, melanoma, prostate cancer, endometrial cancer or a combination thereof.

48. The method of claim 46, wherein said administering is via injection.

49. A method of delaying progression of spontaneous breast tumors in a subject, the method comprising the step of administering to the subject a therapeutically effective dose of said recombinant Listeria of claim 1.

50. The method of claim 50, wherein said administering is via injection.

51. A method of delaying progression of spontaneous breast tumors in a subject, the method comprising the step of administering to the subject a therapeutically effective dose of said recombinant Listeria of claim 1, wherein administering said recombinant Listeria induces epitope spreading to additional tumor associated antigens.

52. The method of claim 50, wherein said administering is via injection.
